(19) United States
(12) Patent Application Publication
(10) Pub. No.: US 2023/0133529 A1
(43) Pub. Date: May 4, 2023

(54) METHOD AND SYSTEM OF PERFORMING CONTROLLED EXPOSURE OF ULTRAVIOLET (UV) RAYS

(71) Applicant: Kohler India Corporation Pvt. Ltd., New Delhi (IN)

(72) Inventors: Yachna Garg, Gurgaon (IN); Atul Dhiman, Noida (IN)

(21) Appl. No.: 17/912,344

(22) PCT Filed: Mar. 17, 2021
(86) PCT No.: PCT/IN2021/050272

§ 371 Date: Sep. 16, 2022

(30) Foreign Application Priority Data
Mar. 19, 2020 (IN) 202011011956

(51) Int. Cl. A61B 5/1171

(52) U.S. Cl. CPC A61B 5/1176; A61N 2005/0627

(57) ABSTRACT

Described herein is a method of controlled exposure of ultraviolet (UV) rays to a user, with use of a UV exposure system comprising a camera unit, a UV source, and a microcontroller. The method includes capturing an image of the user; performing face recognition on the captured image to identify a user profile; obtaining information on dosage value delivered to the user in a defined amount of dosage time period; comparing the dosage value with a threshold value; and performing an action on the UV source based on the comparison. The action includes: activating the UV source if the dosage value is below the threshold value; and deactivating the UV source, if the dosage value is above the threshold value.

### CROSS REFERENCE TO RELATED APPLICATION

This application is a U.S. national stage of International Application No. PCT/IN2021/050272, filed on Mar. 17, 2021, which claims priority to Indian Patent Application No. 202011011956 filed on Mar. 19, 2020, the entire disclosures of which are incorporated by reference herein in its entirety.

### TECHNICAL FIELD

The present disclosure generally relates to a system and a method for performing exposure of ultraviolet (UV) rays to a user. More particularly, the present disclosure relates to the system and method of performing controlled exposure of ultraviolet (UV) rays to the user.

### BACKGROUND

UV radiation is an essential for various health purposes including, such as but not limited to, production of Vitamin ‘D’, regulation of insulin level, diabetes control, supporting lung function, and control of the expression of genes in cancer. When a person is deprived from sufficient amount of ultraviolet (UV) radiations, the person may be subject to rickets, bone weakening, muscle pain, and body aches. Because sunlight is a good source of UV radiations, people are frequently advised by doctors to be exposed to the sunlight for obtaining sufficient amount of ultraviolet (UV) radiations. However, in today's busy era, people do not get sufficient time to be exposed to the sunlight. Therefore, there exists a need for ultraviolet (UV) exposure systems that artificially expose a user to the UV rays, for providing sufficient amount of UV rays to the user.

A UV exposure system is equipped with a UV light source that provides UV rays to the user, upon activation. However, such a UV exposure system is not equipped with a control system to control and deliver an optimum amount of UV rays to the user. In particular, there may be situations when the user may be excessively exposed to the UV rays. In such situations of excessive exposure to the UV rays, there exists many associated risks, including skin cancer, pre-mature aging, skin damage, skin burns, eye damage, and immune suppression. Furthermore, in a method of operating the UV exposure systems, an operator manually activates the UV exposure system to expose the user for a predefined amount of time and thereafter manually deactivates the UV exposure system upon completion of the predefined amount of time. Such manual control of the UV exposure system may be inaccurate and subject to errors. Moreover, in such a UV exposure system, the user is required to dedicate the time to the exposure of the UV rays. Furthermore, when the user wishes to be exposed to the UV rays in a number of small dosages, the operator may cause a manual error in recording the dosage already delivered to the user.

In addition to aforementioned drawbacks of the method of operating the UV exposure system, there is a well felt need of an improved method of operating the UV exposure system to perform controlled exposure of UV rays to the user.

### SUMMARY

One object of the present disclosure relates to a method of performing controlled exposure of ultraviolet (UV) rays to a user. The method comprises: capturing, with use of a camera unit, an image of the user positioned opposite to or in front of a camera unit; performing face recognition on the captured image, with use of a microcontroller, to identify a user profile of the user; obtaining, with use of the microcontroller, information on a dosage value delivered to the user of the identified user profile in a defined amount of dosage time period; comparing, with use of the microcontroller, the dosage value with a threshold value; and performing an action, with use of the microcontroller, on the UV source based on the comparison between the dosage value and the threshold value. The method is initiated upon detection of a presence of a user opposite to or in front of the camera unit, with use of the camera unit. The action performed by the microcontroller includes: activating the UV source, with use of the microcontroller, to expose the user with UV rays when the dosage value is below the threshold value; and deactivating the UV source, with use of the microcontroller, when the dosage value is above the threshold value.

Another object of the present disclosure relates to an ultraviolet (UV) exposure system for performing controlled exposure of the UV rays to a user. The UV exposure system comprises a camera unit, a UV source, and a microcontroller. The camera unit is configured to capture an image of the user positioned opposite to or in front of the camera unit. The UV source is configured to expose the user with UV rays when actuated. The microcontroller is configured to: perform face recognition on the captured image to identify a user profile of the user; obtain information on a dosage value delivered to the user of the identified user profile in a defined amount of dosage time period; compare the dosage value with a threshold value; and perform an action on the UV source based on the comparison between the dosage value and the threshold value. The action performed by the microcontroller includes: activating the UV source, with use of the microcontroller, to expose the user with UV rays when the dosage value is below the threshold value; and deactivating the UV source, with use of the microcontroller, when the dosage value is above the threshold value.

Yet another object of the present disclosure relates to integrally installing of the ultraviolet (UV) exposure system for performing the controlled exposure of the UV rays to the user, onto a furniture unit. The furniture unit is either of a household furniture unit including a mirror, a headboard of a bedding arrangement, and/or a television unit; or a hospital furniture unit, such as but not limited to, an incubator, a hospital bedding arrangement, and/or a hospital headwall. In such systems, each of the camera unit, the UV source, and the microcontroller of the UV exposure system is integrally installed on the furniture unit.

### BRIEF DESCRIPTION OF DRAWINGS

The present disclosure, both as to its organization and manner of operation, together with further objects and advantages, may be understood by reference to the following description, taken in connection with the accompanying drawings. These and other details of the present disclosure are described in connection with the accompanying drawings, which are furnished only by way of illustration and not in limitation of the present disclosure, and in which drawings:

FIG. 1 illustrates a schematic of a household furniture unit implemented with an ultraviolet (UV) exposure system and illustrates various components of the UV exposure system according to an embodiment of the present disclosure.

FIG. 2 illustrates a flowchart of a method of performing controlled exposure of ultraviolet (UV) rays to a user, as employed by the UV exposure system of FIG. 1, according to an embodiment of the present disclosure.

### DETAILED DESCRIPTION

In the following description, for the purposes of explanation, various specific details are set forth in order to provide a thorough understanding of embodiments of the present disclosure. It should be apparent, however, that embodiments of the present disclosure may be practiced without these specific details. Several features described hereafter can each be used independently of one another or with any combination of other features. Embodiments of the present disclosure are described below, as illustrated in various drawings in which like reference numerals refer to the same or equivalent parts throughout the different drawings.

The present disclosure discloses an ultraviolet (UV) exposure system 102 for performing controlled exposure of the UV rays to users. Furthermore, the present disclosure also discloses a method 104 employed by the UV exposure system 102, for performing controlled exposure of the UV rays to the users. Moreover, the present disclosure discloses a furniture unit 100 that integrally installs the UV exposure system 102 for performing controlled exposure of the UV rays to the users. Although the present disclosure hereinafter describes the furniture unit 100 and integral installation of the UV exposure system 102 on the furniture unit 100, it should be apparent to a person having ordinary skill in the art that the concepts of the present disclosure may also extend to a bathing unit and integral installation of the UV exposure system 102 on the bathing unit. In particular, integral installation of the UV exposure system 102 on the bathing unit also lies within a scope of the present disclosure, wherein the bathing unit may include a bathing shower, a bathing tub, a jacuzzi, and/or a bathing cabinet. For ease in reference and understanding, while concepts of the present disclosure are described as applied to the furniture unit 100 and installation of the UV exposure system 102 on the furniture unit 100, similar concepts of the present disclosure may be applied to the bathing unit and installation of the UV exposure system 102 on the bathing unit.

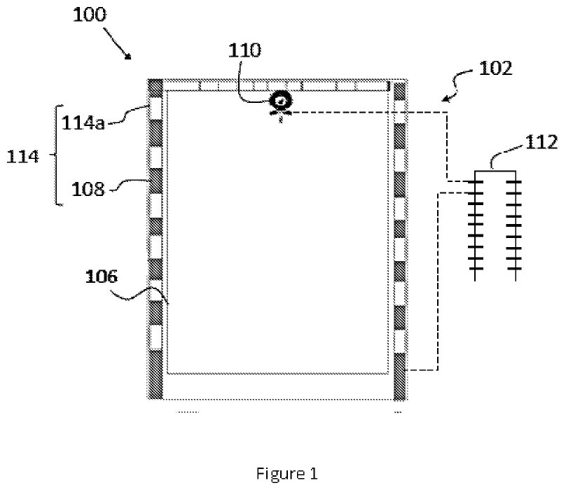

FIG. 1 shows a schematic of the furniture unit 100 employing the ultraviolet (UV) exposure system 102, for performing controlled exposure of the UV rays to the users. The UV exposure system 102 is integrally installed on the furniture unit 100, to facilitate controlled exposure of the UV rays to a user, while the user uses the furniture unit 100. In an embodiment, the furniture unit 100 may include any household furniture unit, such as but not limited to, a headboard of a bedding arrangement, a vanity mirror, a study table, a kitchen cabinet, and the like. With such embodiments of the furniture unit 100, the users are able to perform a household task and concurrently get exposed to the UV rays. In another embodiment, the furniture unit 100 may include any hospital furniture unit, such as but not limited to, an incubator, a hospital bedding arrangement, a hospital headwall, and the like. Concepts of the present disclosure hereinafter are defined as UV exposure system 102 integrally installed onto the vanity mirror 100 as an embodiment. However, it should be apparent to a person having ordinary skill in the art that the concepts of the present may also be applied to the UV exposure system 102 integrally installed on other aforementioned examples of the furniture unit 100. For ease in reference and understanding, the furniture unit 100 may be referred to as the vanity mirror 100, interchangeably hereinafter.

The vanity mirror 100 may be used by a user to apply make-ups or get ready for a party, and the like. The vanity mirror 100 may be any silver polished mirror that reflects an image of a user to be viewed by the user. The vanity mirror 100 may include any shape, profile, and structure, defining a peripheral portion 106.

The UV exposure system 102 is integrally installed on the vanity mirror 100. Specifically, as the vanity mirror 100 is frequently used by users, integrally installing the UV exposure system 102 onto the vanity mirror 100 corresponds to relatively promised exposure of the UV rays to the users. The UV exposure system 102 includes a UV source 108, a camera unit 110, and a microcontroller 112.

The UV source 108 is a part of a light unit 114 installed along the peripheral portion 106 of the vanity mirror 100. In particular, the light unit 114 has alternate portions of a light source 114a and the UV source 108. The light source 114a outputs visible light to be projected onto the user's body using the vanity mirror 100, for improved illumination. The visible light outputted by the light source 114a is within a range of 400-700 nm. The UV source 108 outputs UV light to output the UV rays be projected onto the user's body for exposure to the UV rays. The UV rays outputted by the UV source 108 to be exposed to the user is within a range of 280-320 nm. Each of the UV source 108 and the light source 114a may be controlled for activation and/or deactivation by the microcontroller 112, as and when required.

The camera unit 110 is positioned on a top center position of the vanity mirror 100. The camera unit 110 is configured to detect a presence of a user positioned in front of the vanity mirror 100. The camera unit 110 is suitably positioned to capture images of a user positioned opposite to or in front of the camera unit 110 (and thus opposite to or in front of the vanity mirror 100). It may be noted that the camera unit 110 is suitably positioned on the vanity mirror 100, such that the camera unit 110 focuses on a single user and captures the images of the single user, even when multiple users are positioned opposite to or in front of the camera unit. The camera unit 110 may further be controlled by the microcontroller 112, for capturing the images of the user positioned in front of the vanity mirror 100.

The microcontroller 112 is electrically connected to each of the light source 114a and the UV source 108 of the light unit 114 and the camera unit 110, to control each of the light source 114a, the UV source 108, and the camera unit 110, in accordance to a method 104 of performing controlled exposure of the UV rays to the user as disclosed in the present disclosure. The microcontroller 112 may be a combination of a memory unit, a processor, and a number of other electronic components, capable of performing the method 104 of controlled exposure of the UV rays to the user. The microcontroller 112 may be either of an 8-bit microcontroller, 16-bit microcontroller, a 32-bit microcontroller, and/or a 64-bit microcontroller. Examples of the microcontroller 112 include, such as but not limited to, an 8081 microcontroller, an 8085 microcontroller, a PIC2x, an Intel 8096, and/or MC68HC12 families.

The microcontroller 112 is configured to perform the method 104 of performing controlled exposure of the UV rays to the user. For such purposes, the microcontroller 112 stores an information on a dosage value delivered to a number of users corresponding to a number of user profiles in a defined amount of dosage time period. In particular, the microcontroller 112 has the database that stores information for a number of users, including a user profile (for example a user profile ID) and a dosage value delivered in a defined amount of dosage time period, for each user. The dosage time period is a time period between a preset time instance and a current time instance. In an embodiment, the microcontroller 112 may be programmed to define 00:00 A.M. as the preset time instance, for storing the dosage value delivered to the users in a single day time period. In such embodiments, the dosage time period is the time period between the start of the day instance and the current time instance, for each user. With such storage of information on user profile (for example a user profile ID) and the dosage value delivered in the dosage time period, the microcontroller 112 is capable of performing the method 104 of performing controlled exposure of the UV rays to the users. In particular, the microcontroller 112 is programmed and configured to perform the following: perform face recognition on the captured image to identify a user profile of the user; obtain a dosage value delivered to the user of the identified user profile in a defined amount of dosage time period; compare the dosage value with a threshold value; and perform an action on the UV source 108 based on the comparison between the dosage value and the threshold value. Moreover, the microcontroller 112 performs actuation of the UV source 108 when the dosage value is below the threshold value and performs deactivation of the UV source 108 when the dosage value is above the threshold value. ‘Activation’ of the UV source 108 herein refers to adjusting the UV source 108 to an ‘ON’ mode, in which the UV source 108 exposes the user with UV rays. ‘Deactivation’ of the UV source 108 herein refers to adjusting the UV source 108 in an ‘OFF’ mode, in which the UV source 108 stops exposing the user with UV rays. Moreover, ‘Deactivation’ of the UV source 108 herein refers to adjusting the UV source 108 in the ‘OFF’ mode if the UV source 108 is previously in the “ON’ mode; and keeping the UV source 108 in the ‘OFF’ mode if the UV source 108 is previously in the OFF’ mode.

The method 104 implemented by the microcontroller 112 of the UV exposure system 102 may perform the controlled exposure of ultraviolet (UV) rays to users. Although the present disclosure describes the threshold value as a prestored value that is constant for all user profiles, it should be apparent to a person having ordinary skill in the art that the threshold value may be a value dependent on the user profile, i.e., each user profile may have a different threshold value for each user. Moreover, the threshold value may also be a user-customizable value in place of the prestored value, which can be changed if required.

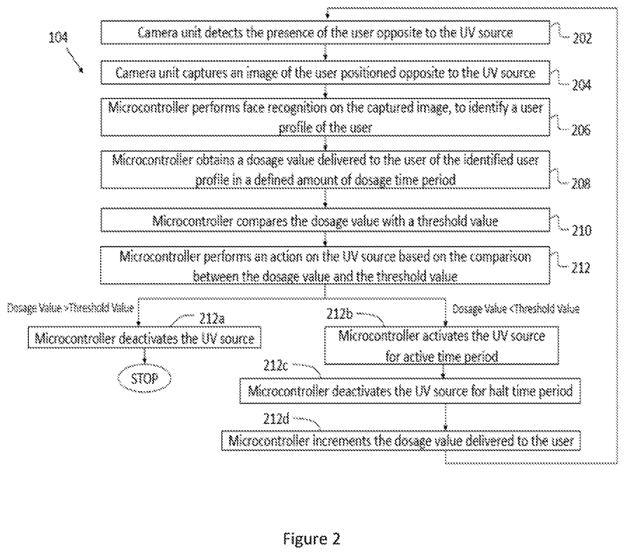

FIG. 2 shows a flowchart of the method 104 of performing the controlled exposure of ultraviolet (UV) rays to the users. The method 104 may be initiated at step 202. At step 202, as a user steps in front of the vanity mirror 100, the camera unit 110 detects the presence of the user opposite to or in front of the camera unit 110. The camera unit 110, upon detection of the presence of the user opposite to or in front of the camera unit 110, signals the microcontroller 112 to initiate the method 104 of performing the controlled exposure of ultraviolet (UV) rays to the user. The method 104 then proceeds to step 204.

At step 204, the microcontroller 112 sends a signal to the camera unit 110, to capture an image of the user positioned opposite to or in front of the camera unit 110. The camera unit 110 is suitably positioned on the vanity mirror 100 to capture the image of a single user positioned opposite to or in front of the camera unit 110. Therefore, when multiple users are positioned opposite to or in front of the camera unit 110, the camera unit 110 captures the image of the single user positioned opposite to or in front of the camera unit 110. The camera unit 110 transfers the captured image to the microcontroller 112. The method 104 then proceeds to step 206.

At step 206, the microcontroller 112 performs face recognition on the captured image, to identify a user profile of the user whose image is captured. In particular, the microcontroller 112 runs the face recognition algorithm to identify the user profile of the user whose image is captured. Specifically, the microcontroller 112 stores algorithm for face recognition algorithm to perform the face recognition on the captured image. Furthermore, the microcontroller 112 also has a database of user database that stores information for a number of users, including a user profile (for example a user profile ID) and a dosage value delivered in a defined amount of dosage time period, for each user. At this step, the microcontroller 112 performs face recognition relative to the entire database, to identify a user profile of the user positioned opposite to or in front of the camera unit 110. The method 104 then proceeds to step 208.

At step 208, the microcontroller 112 obtains a dosage value delivered to the user of the identified user profile in a defined amount of dosage time period. The defined amount of dosage time period is a time period between a preset time instance and a time instance during initiation of the method 104. In an embodiment, the preset time instance is 00:00 A.M. Therefore, in such embodiments, the defined amount of dosage time period defines the time period elapsed in the day of performing the method 104 until the method 104 is initiated. Moreover, the dosage value obtained at step 208, in such embodiments, is the dosage delivered to the user of the identified user profile during the same day until the method 104 is initiated. The method 104 then proceeds to step 210.

At step 210, the microcontroller 112 compares the dosage value with a threshold value. In particular, the microcontroller 112 identifies if the dosage value delivered to the user of the identified user profile in the defined amount of dosage time period is above/below the threshold value. The method 104 then proceeds to step 212.

At step 212, the microcontroller 112 performs an action on the UV source 108 based on the comparison between the dosage value and the threshold value. In particular, based on the comparison, the method 104 proceeds to either of step 212a or step 212b. At step 212a, the microcontroller 112 deactivates the UV source 108 when the dosage value is above the threshold value. After step 212a, the method 104 is terminated. Moreover, at step 212b, the microcontroller 112 activates the UV source 108 to expose the user with UV rays when the dosage value is below the threshold value. In particular, the microcontroller 112 activates the UV source 108 for at least a defined amount of active time period. After the defined amount of active time period, the method 104 proceeds to step 212c. At step 212c, the microcontroller 112 deactivates the UV source 108 for at least a defined amount of halt time period. After step 212c, the method 104 proceeds to step 212d. At step 212d, the microcontroller 112 increments the dosage value delivered to the user of the identified user profile. After step 212d, the method 104 proceeds to perform step 202 again.

It may be noted that as the method 104 disclosed in the present disclosure may deactivate the UV source 108 when the dosage value reaches above the threshold value, the method 104 may limit the exposure of the user to the UV rays from the UV source 108 up to the threshold value. Therefore, over-exposure of the UV rays from the UV source 108 may be avoided. Moreover, as the method 104 disclosed in the present disclosure activates the UV source 108 for the defined amount of active time period only and deactivates thereafter, the method 104 may avoid over-exposure of the user to the UV rays from the UV source 108. Furthermore, as the method 104 provides the UV rays exposure to the user by the UV source 108 within a range of 280 nm-320 nm, such range is relatively safer and provides for safe exposure of the user to the UV rays by the UV source 108. Moreover, as the method 104 and the ultraviolet (UV) exposure system 102 are integrally installed on the furniture unit 100, such an arrangement provides exposure of the users to the UV rays by the UV source 108 while the user uses the furniture unit 100. In particular, in one embodiment, the method 104 and the ultraviolet (UV) exposure system 102 is integrally installed on the vanity mirror 100. Therefore, such an arrangement of the method 104 and the ultraviolet (UV) exposure system 102 with the vanity mirror 100 provides controlled exposure of the UV rays by the UV source 108 of the ultraviolet (UV) exposure system 102 and concurrent use of the vanity mirror 100. In nutshell, such an arrangement provides controlled exposure of the UV rays from the UV source 108 to the user while using the vanity mirror 100. Similar to the embodiment of the vanity mirror 100, in an alternated embodiment, the method 104 and the ultraviolet (UV) exposure system 102 may be integrally installed on a bathing shower. Therefore, such an arrangement of the method 104 and the ultraviolet (UV) exposure system 102 with the vanity mirror 100 provides controlled exposure of the UV rays by the UV source 108 of the ultraviolet (UV) exposure system 102 and concurrent use of the bathing shower for bathing purposes. In nutshell, such an arrangement provides controlled exposure of the UV rays from the UV source 108 to the user while the user uses the bathing shower.

Further, it may be noted that as an ultraviolet (UV) exposure system 102 and the method 104 as disclosed in the present disclosure may perform controlled exposure of ultraviolet (UV) rays to a user. With such exposure to the UV rays by use of the ultraviolet (UV) exposure system 102 and the method 104 as disclosed herein, the user may produce enough Vitamin ‘D’, which provides improved bone strength, better regulation of insulin level, diabetes control, improved lung function support, and controlled expression of genes in cancer. This avoids chances of rickets, bone weakening, and muscle pain to the user. It may further be noted that the ultraviolet (UV) exposure system 102 and the method 104 as disclosed in the present disclosure provides controlled exposure of the user to the UV ray, while the user performs other household work of using the furniture unit 100. In particular, the user may use the furniture unit 100 (for example may use the vanity mirror unit 100) and concurrently get exposed to the UV rays by the disclosed UV exposure system 102 and the method 104. By doing so, the user may also be prevented from over-exposure of the UV rays, and thus the user may be prevented from skin cancer, pre-mature aging, skin damage, skin burns, eye damage, and immune suppression.

While the embodiments of the present disclosure have been described hereinabove, it should be understood that various changes, adaptations, and modifications may be made therein without departing from the spirit of the present disclosure and the scope of the appended claims. It should be apparent to a person having ordinary skill in the art that the present disclosure may be embodied in other specific forms without departing from its spirit or essential characteristics. The described embodiments should be considered in all respects only as illustrative and not restrictive.

### LIST OF COMPONENTS

100— Furniture Unit
100— Vanity Mirror
102— UV Exposure System
104— Method
106— Peripheral Portion of 100
108— UV Source of 102
110— Camera Unit of 102
112— Microcontroller of 102
114— Light Unit
114a— Light Source
202-212—Steps of 104
212a-212d— Sub-steps of 212

1-17. (canceled)

18. A method of performing controlled exposure of ultraviolet (UV) rays to a user, the method comprising:
capturing, by a camera unit, an image of the user in front of the camera unit;
performing face recognition on the captured image, by a microcontroller, to identify a user profile of the user;
obtaining, by the microcontroller, a dosage value delivered to the user of the identified user profile in a defined amount of dosage time period;
comparing, by the microcontroller, the dosage value with a threshold value;
performing an action, by the microcontroller, on a UV source based on the comparison between the dosage value and the threshold value.

19. The method according to claim 18, further comprising:
detecting, by the camera unit, a presence of a user in front of the camera unit to initiate the method.

20. The method according to claim 18, wherein performing the action on the UV source comprises:
activating the UV source, by the microcontroller, to expose the user with the UV rays when the dosage value is below the threshold value, and
deactivating the UV source, by the microcontroller, when the dosage value is above the threshold value.

21. The method according to claim 18, wherein the defined amount of dosage time period is a time period between a preset time instance and a time instance during initiation of the method.

22. The method according to claim 18, wherein the exposure of the user to the UV rays from the UV source is limited to the threshold value.

23. The method according to claim 20, wherein the UV source is kept activated for at least a defined amount of active time period.

24. The method according to claim 23, wherein the UV source is deactivated for at least a defined amount of halt time period after performing the activation for at least the defined amount of active time period.

25. The method according to claim 18, wherein the microcontroller increments the dosage value delivered to the user and stores the incremented dosage value, after the UV source is activated for a defined amount of active time period.

26. The method according to claim 18, wherein the UV rays exposed to the user by the UV source is within a range of 280 nm-320 nm.

27. An ultraviolet (UV) exposure system for performing controlled exposure of UV rays to a user, the UV exposure system comprising:
a camera unit configured to capture an image of the user in front of the camera unit;
a UV source configured to expose the user with the UV rays when the UV source is actuated; and
a microcontroller configured to:
perform face recognition on the captured image to identify a user profile of the user;
obtain a dosage value delivered to the user of the identified user profile in a defined amount of dosage time period;
compare the dosage value with a threshold value; and
perform an action on the UV source based on the comparison between the dosage value and the threshold value.

28. The ultraviolet (UV) exposure system according to claim 27, wherein the microcontroller performs actuation of the UV source when the dosage value is below the threshold value and performs deactivation of the UV source when the dosage value is above the threshold value.

29. The ultraviolet (UV) exposure system according to claim 27, wherein the defined amount of dosage time period is a time period between a preset time instance and a time instance during initiation capturing of image by the camera unit.

30. The ultraviolet (UV) exposure system according to claim 27, wherein the ultraviolet (UV) exposure system is integrally installed on a furniture unit.

31. The ultraviolet (UV) exposure system according to claim 30, wherein the furniture unit includes a vanity mirror, a study table, a headboard of a bedding arrangement, and/or a television unit.

32. The ultraviolet (UV) exposure system according to claim 31, wherein each of the camera unit, the UV source, and the microcontroller of the UV exposure system are integrally installed on the vanity mirror when the UV exposure system is integrally installed on the vanity mirror.

33. The ultraviolet (UV) exposure system according to claim 32, wherein the vanity mirror comprises a plurality of light sources and the UV exposure system comprises a plurality of UV sources, and
wherein the plurality of light sources and the plurality of UV sources are alternately disposed along a peripheral portion of the vanity mirror.

34. The ultraviolet (UV) exposure system according to claim 27, wherein the ultraviolet (UV) exposure system is integrally installed on a bathing unit.

35. The ultraviolet (UV) exposure system according to claim 34, wherein the bathing unit includes a bathing shower, a bathing tub, a jacuzzi, and a bathing cabinet.

36. The ultraviolet (UV) exposure system according to claim 27, further comprising:
a database configured to store information for a plurality of users, including a user profile and a dosage value delivered in a defined amount of dosage time period for each user of the plurality of users,
wherein the microcontroller is configured, by accessing the information stored in the database, to perform the face recognition on the captured image to identify the user profile of the user in front of the camera unit, and
wherein the threshold value depends on the user profile of each user of the plurality of users.

37. The ultraviolet (UV) exposure system according to claim 36, wherein the camera unit is further configured to focus on a single user between or among the multiple users and captures the images of the single user when the multiple users are in front of the camera unit.
